(19) United States
(12) Patent Application Publication
(10) Pub. No.: US 2008/0032345 A1
(43) Pub. Date: Feb. 7, 2008

(54) Method and Tools for Analysis of Nucleic Acids

(76) Inventors: Janusz Fiett, Warszawa (PL); Krzystof Kucharczyk, Warzsawa (PL)

Correspondence Address: COOPER & DUNHAM, LLP, 1185 AVENUE OF THE AMERICAS, NEW YORK, NY, 10036, US

(21) Appl. No.: 11/578,642

(22) PCT Filed: Apr. 26, 2005
(86) PCT No.: PCT/PL05/00027

§ 371 Date: Aug. 2, 2007

(30) Foreign Application Priority Data
Apr. 26, 2004 (PL) P.367570

(51) Int. Cl. C12P 19/34; C12M 1/00; C12N 5/06

(52) U.S. Cl. 435/91.1; 435/285.1; 435/375

(57) ABSTRACT

A method and tools for the analysis of nucleic acids has been disclosed, in particular the present invention relates to the isolation of genomic DNA from cells and its enzymatic modification. The subjects of the present invention are used to analyse the genetic material of organisms. e.g. to establish the relationship between the studied organisms.

The subject of the present invention is a method and tools for the analysis of nucleic acids. In general, the present invention relates to the isolation of genomic DNA from cells and its enzymatic modification. The subjects of the present invention are used to analyse the genetic material of organisms. e.g. to establish the relationship between the studied organisms.

Nucleic acid analysis methods may be used in the identification or differentiation of organisms. A common method of analysing genomic DNA is PFGE, Pulsed Field Gel Electrophoresis. This method is used in the analysis of large molecules/fragments of DNA based on their size. The difference between PFGE and classic agarose gel separation of DNA fragments is the application of an alternating, not steady, electric potential gradient vector. In contrast to steady electric field electrophoresis, which may differentiate DNA fragments of up to several tens of thousands nucleotide pairs long, PFGE may differentiate molecules even several million nucleotide pairs long. PFGE is used in the analysis and preparation of DNA molecules of large size, such as eukaryotic and bacterial chromosomes, large plasmids or DNA fragments resulting from the digestion of entire genomes with rarely cleaving restriction endonucleases. This is used in the characterisation of genomic structure, the identification of specific genetic structures within the genome, the isolation of particular portions of the genome, or in relationship analyses between organisms.

PFGE turned out to be particularly useful in the epidemiological typing of pathogenic microbes using Restriction Fragment Length Polymorphism method (RFLP).

DNA molecules prepared for PFGE analysis are from several tens of thousands to several million base pairs long. DNA molecules that long, when obtained using traditional preparative methods in solutions are mechanically damaged (cleaved) by hydrodynamic forces, which renders them useless for further analysis using PFGE. In order to limit this type of DNA damage, the initial biological material is imbedded in agarose, formed into a block. Such block of agarose with whole cells inside is incubated in a series of steps, of particular duration and temperature, in solutions appropriate to the biological material. These contain chemical factors, enzymes and inhibitors, which diffuse into the interior of the block and cause cell lysis, protein and RNA hydrolysis, as well as the inactivation of cell enzymes which could cause the genomic DNA to degrade. In order to increase the rate of diffusion of factors from the solutions into the block, as well as of freed substances out of the block, a mixing of solutions is used. In most applications, the last step in the preparation of the DNA for PFGE is its digestion with a restriction endonuclease. Due to the size of DNA molecules separated using PFGE, the success of the experiments is particularly badly threatened by external deoxyribonucleases. The preparation of the DNA must occur in conditions which minimize contamination, inclusive of nucleases. This is why it is vital that all manipulation of the blocks as well as solution changes be performed as rapidly as possible in single-use containers.

In commercial kits for the preparation of PFGE DNA, special forms are included into which a suspension of biological material in agarose of an appropriate temperature is poured. After setting, the formed oblong blocks are removed using a special tool. At this time, it is common practice to prepare multiple replicants of the blocks for each sample. This is justified by the common practice of using different restriction endonucleases, or the necessity to repeat the electrophoretic separation arising for technical reasons or due to the complexity of the experiment. Blocks containing the purified DNA may be stored in an appropriate solution for as long as several months at a decreased temperature. Alternative methods for preparing agarose blocks for PFGE analysis are also known. The first consists of applying droplets of agarose/biological material mixture onto glass slides coated with a hydrophobic substance, and covering them with slips placed on appropriate supports. After setting, this forms blocks in the shape of flattened disks. The second alternative method consists of forming a cylindrical block of agarose and biological material inside a syringe with the tapering portion removed. Subsequently, the syringe plunger is used to propel the agarose block out of the barrel, to be cut into thin disks. All blocks containing a given sample of biological material are jointly placed in a closed container. The DNA purification procedure consist of a series of incubations in appropriate solutions, maintaining time, temperature and possibly mixing. All procedures involved in transferring the blocks between containers (e.g. for digestion with restriction endonucleases) as well as placing them in agarose gels are performed manually and require great dexterity and precision. Tools for standardising or facilitating these complex procedures are unavailable.

The first amelioration of this process was the placement of a sieve under the lid of screw-cap, plastic tubes typically used in the laboratory. The sieve facilitates the exchange of incubation solutions, without the danger of losing the blocks. The most commonly used method of isolating DNA for PFGE is based on manually preparing a series of agarose blocks with embedded biological material. These are placed in test-tubes of which each contains the blocks containing one particular type of sample. The solutions in the tubes are subsequently exchanged manually. During this procedure, it is necessary to take particular care not to mechanically damage the agarose blocks. Methods of isolation and analysis of genomic DNA may be classified as:

1. Classical methods. There is a range of types and/or modifications of genomic DNA isolation. One of the most commonly used was phenol/chloroform extraction (Molecular Cloning, a laboratory manual, second edition; J. Sambrook, E. F. Fritsch, T. Maniatis; Cold Spring Harbor Laboratory Press, 1989, part E.3). Phenol was used to denature proteins, and the chloroform was used to purify the genomic DNA. These methods, due to the need to transfer and mix solutions containing the genomic DNA cause a large number of cleavages in said DNA. It is practically impossible to obtain whole, intact genomic DNA in this way.

2. Isolation of genomic DNA using affinity DNA purification on silica beds. This is the most common, commercial genomic DNA purification method. However, like in method 1 the necessity of several transfers and mixing of solutions containing genomic DNA, this leads to the exertion of hydrodynamic forces and cleaving. It is practically impossible to obtain whole, intact genomic DNA in this way.

3. Isolation of genomic DNA in agarose blocks, as described above. This is the only procedure which facilitates the isolation of genomic DNA as intact molecules. Its significant drawback is the need to manually perform a large number of operations in many replicants, which absorbs many man hours of qualified workers' time.

The methods described above have expanded the knowledge and methodology of the isolation and analysis of genomic DNA. To date, however, tools and methods for automating the preparation of genomic DNA for PFGE separation remain unavailable, which would make the method less labour-intensive, shorter and make the results more repeatable. This would then allow PFGE to be more widely applied in routine medical diagnostics, as well as shortening the procedure time, something of great importance in epidemiological studies.

The goal of the present invention is to deliver the tools and methods which could be used in the effective isolation of nucleic acids from cells, particularly DNA molecules for PFGE analysis, and particularly for the purification and restriction endonuclease digestion of genomic DNA from bacteria responsible for epidemiological events.

Unexpectedly, the embodiment of such a described goal and the solution of the problems with the isolation of large nucleic acid molecules described in the state of the art have been resolved in the present invention.

The subjects of the present invention are a method and a device for the isolation and modification of nucleic acids from biological, characterised in that they facilitate the purification of DNA (e.g. genomic DNA) from biological material contained in agarose blocks as well as its enzymatic modification (e.g. modification with restriction endonucleases), through the contact of the block with appropriate solutions, under controlled conditions. The interchangeably used terms “isolation” and “purification” denote the process, during which the percent composition of DNA significantly grows among substances originating from a cell within the agarose block.

Preferentially, the exchange of solutions, their agitation and changes in solution temperature occur automatically according to a program determined by the user.

Preferentially, a portion of the blocks with the biological material indicated within the program may be arbitrarily selected for restriction endonuclease digestion of the purified DNA.

Preferentially, the solutions and other substances necessary for the procedure can be delivered by the user, prior to the realisation of the procedure by the device.

Preferentially, the device facilitates the storage of solutions and other substances necessary for the procedure and provided by the user, in pre-programmed temperatures.

Preferentially, all of the elements of the device which come into contact with solutions used to incubate the blocks are single-use elements.

The next subject of the present invention is an element, henceforth called the array, which is comprised of a solid medium to which blocks containing biological material are attached.

Preferentially, the array also facilitates the storage, transfer and incubation of blocks containing biological material of various origins in the same solutions.

Preferentially, the array is in the form of a plate with holes, characterised in that the size and shape of apertures ensure the formation blocks; they ensure their adhesion to the solid medium during the procedures during the purification and modification of the DNA such that a majority of the blocks have contact with the external environment, and such that the blocks are easy to remove.

Preferentially, the shape or texture of the aperture walls ensures adequate adhesion of the blocks to the array.

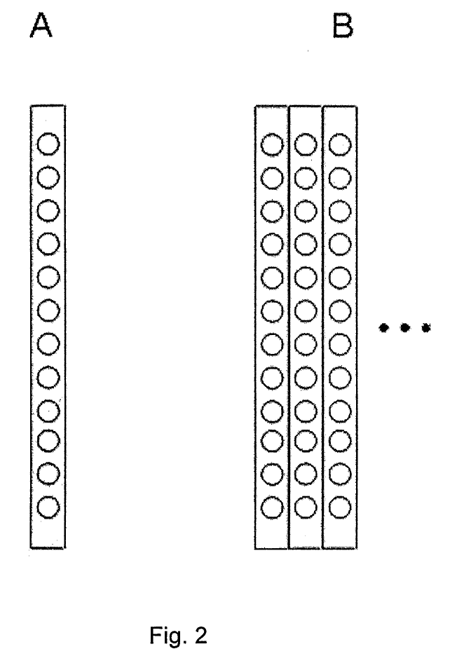

Preferentially, the shape of the array according to the present invention corresponds with the element from FIG. 2A.

Preferentially, several arrays are bound into a multiple array corresponding to the element in FIG. 2B Preferentially, the array is made through injection moulding.

Preferentially, the array is single-use disposable.

Preferentially, blocks containing different biological material are placed in individual apertures of a single array.

Preferentially, blocks containing the same biological material are placed in corresponding apertures of arrays bound in a multiple array.

The next subject of the present invention is an application of the device and of the array in the simultaneous isolation of genomic DNA from a series of biological samples.

The enclosed Figures aid in the more complete explanation of the nature of the present invention.

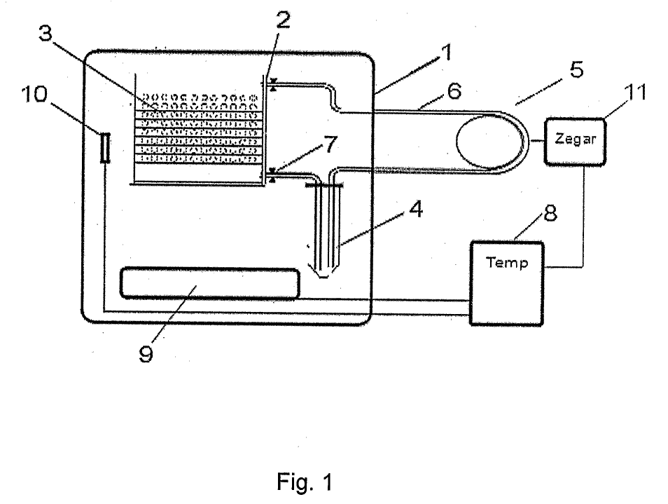

FIG. 1 represents a block schematic of an example device according to present invention.

Element No. 1, is thermal shielding of the incubation chamber. Element No. 2, is the incubation chamber of the agarose block array. Element No. 3, is the array, to which are bound agarose fragments with biological material. Element No. 4, is the container for the storage and exchange of incubation solutions. Element No. 5, is a pump which is transferring the incubating solutions for the agarose blocks. Element 6, are the hoses connecting the incubation chamber (2) with the pump (5) the buffer storage/exchange chamber (4). Element 7, is a valve which halts buffer-flow from the chamber (2). Element 8, is a temperature regulatory system. Element 9, is the heating/cooling module (8). Element 10, is a thermal sensor. Element 1, is a timer controlling the activity of the pump (5) and temperature regulator (8).

FIG. 2 represents an array according to the present invention. Portion A of the picture represents a single array. This array may be made of plastic. In the solution presented, the apertures in which the biological material is contained are circular. These apertures may have any arbitrary shape, regular or irregular.

Portion B of the picture represents single arrays which have been joined to form a multiple array, which facilitates the parallel isolation and modification of DNA in a larger number of blocks, containing cells from various sources, or likewise the isolation and modification of DNA from a single source of material in many replicants.

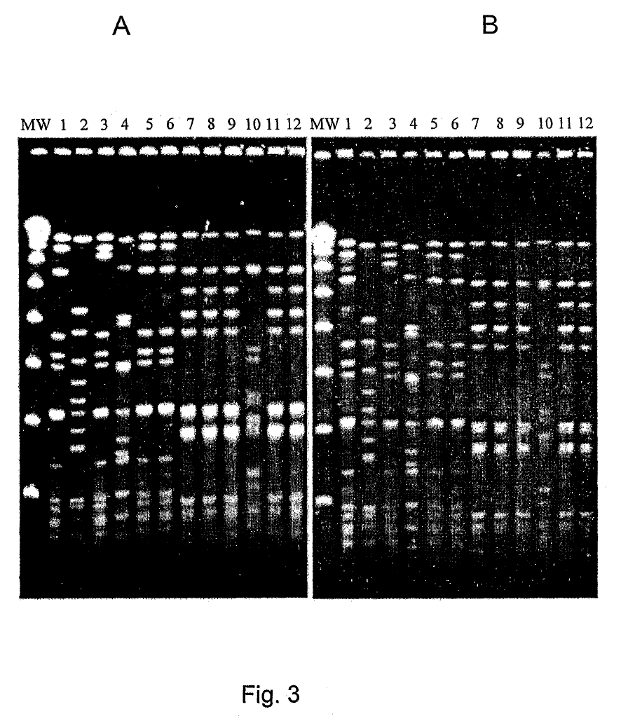

FIG. 3 compares the genotyping results of 12 strains of Haemophilus influenzae using PFGE. In both panels (A and B), the leftmost lane, designated MW, contains DNA mass markers.

Panel A represents a gel image, in which lanes 1 to 12 contain PFGE-separated genomic DNA fragments obtained using the method according to the present invention, stained with ethidium bromide and illuminated with UV.

Panel B represents a gel image, in which lanes 1 to 12 contain PFGE-separated genomic DNA fragments obtained using the classical method of rinsing and incubation of biological sample-containing agarose blocks in various solutions, in test tubes. After elecrophoresis, the gels were stained with a solution of ethidium bromide and illuminated with UV.

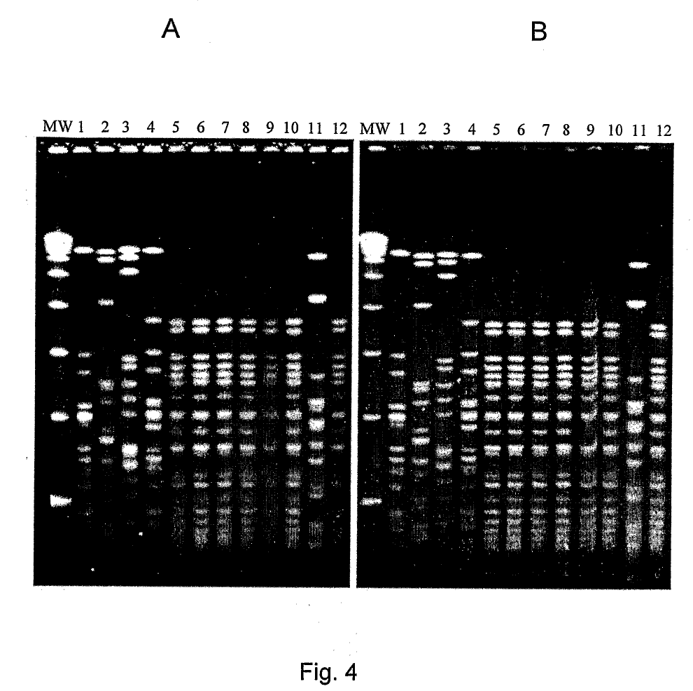

FIG. 4 compares the genotyping results of 12 strains of Neisseria meningitidis using PFGE. In both panels (A and B), the leftmost lane, designated MW, contains DNA mass markers.

Panel A represents a gel image, in which lanes 1 to 12 contain PFGE-separated genomic DNA fragments obtained using the method according to the present invention, stained with ethidium bromide and illuminated with UV.

Panel B represents a gel image, in which lanes 1 to 12 contain PFGE-separated genomic DNA fragments obtained using the classical method of rinsing and incubation of biological sample-containing agarose blocks in various solutions, in test tubes. After elecrophoresis, the gels were stained with a solution of ethidium bromide and illuminated with UV.

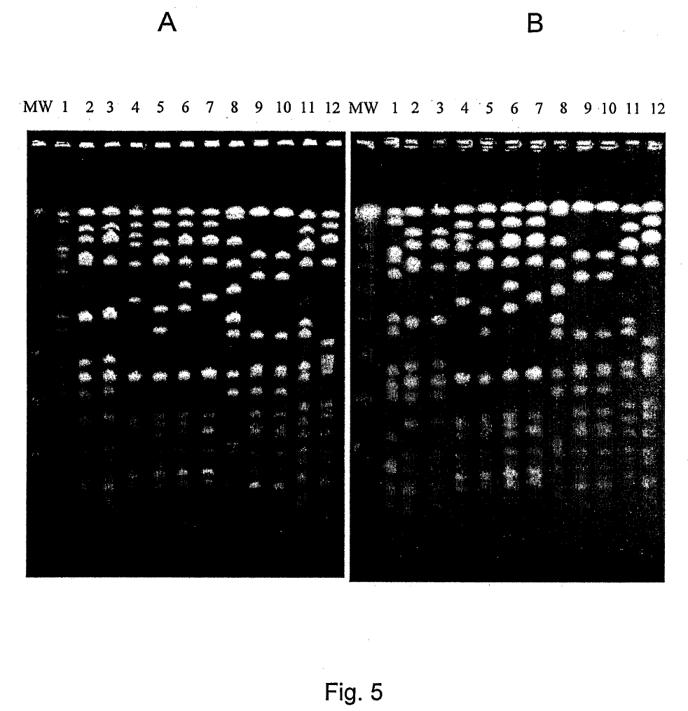

FIG. 5 compares the genotyping results of 12 strains of Staplylococcus aureus using PFGE. In both panels (A and B), the leftmost lane, designated MW, contains DNA mass markers.

Panel A represents a gel image, in which lanes 1 to 12 contain PFGE-separated genomic DNA fragments obtained using the method according to the present invention, stained with ethidium bromide and illuminated with UV.

Panel B represents a gel image, in which lanes 1 to 12 contain PFGE-separated genomic DNA fragments obtained using the classical method of rinsing and incubation of biological sample-containing agarose blocks in various solutions, in test tubes. After elecrophoresis, the gels were stained with a solution of ethidium bromide and illuminated with UV.

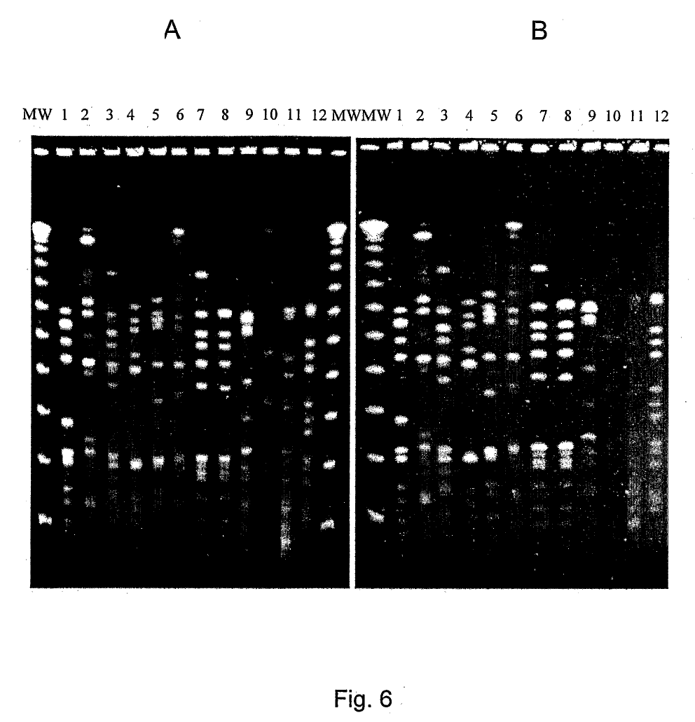

FIG. 6 compares the genotyping results of 12 strains of Streptococcus pneumoniae using PFGE. In both panels (A and B), the leftmost lane (and in panel A also the rightmost lane), designated MW, contains DNA mass markers.

Panel A represents a gel image, in which lanes 1 to 12 contain PFGE-separated genomic DNA fragments obtained using the method according to the present invention, stained with ethidium bromide and illuminated with UV.

Panel B represents a gel image, in which lanes 1 to 12 contain PFGE-separated genomic DNA fragments obtained using the classical method of rinsing and incubation of biological sample-containing agarose blocks in various solutions, in test tubes. After elecrophoresis, the gels were stained with a solution of ethidium bromide and illuminated with UV.

Below, are example embodiments of the present invention.

### EXAMPLE 1

FIG. 1 represents a schematic cross-section of a device for the parallel incubation of a series of biological samples embedded in agarose blocks as an example of the present invention.

The example device is composed of:

1. A thermal shield whose function is to ensure constant temperature for the samples during the stages of the process.

2. An incubation chamber, which facilitates the simultaneous incubation of a series of samples attached to the array (3). The device can be equipped with several such chambers 2a, 2b etc., into which the array or multiple array (3) of analysed material will be transferred mechanically or manually.

3. The array, to which are bound the agarose fragments containing the biological samples.

4. A chamber for storing and exchanging the incubation solutions.

5. A pump for pumping the incubation solutions

6. Hose connecting the pump (5) with the incubation chamber (2)

7. A valve which halts solution out-flow from the incubation chamber (2) when closed

8. The temperature control system, composed of a temperature sensor (10), a heating/cooling element (9), a measuring/regulating system (8), and a control module (11).

The main modules comprising the device for the simultaneous incubation of a series of biological samples are: the incubator, composed of incubation chamber (2) or several chambers (2a, 2b, 2c itd.) as well as the array or a multiple arrays for holding the blocks with biological material (3), chamber or chambers (4) for storing and exchanging incubation solutions, a temperature sensor (10), a heating/cooling element (9) and a pump (5) facilitating the exchange of solutions and their motion within the incubation chamber. The first to be described will be the elements and procedures necessary for the isolation of genomic DNA from cells, and then the procedures and tools necessary for the digestion of genomic DNA with selected restriction endonucleases.

One side of the array (3) is sealed with e.g. adhesive tape such that each aperture becomes an independent and water-tight chamber. An array prepared in this way is placed on a horizontal surface, and melted agarose is poured into each of the apertures. Before the agarose is poured into apertures the biological material from which DNA is to be isolated is added to the agarose. After the agarose sets, the adhesive tape is removed from the array. Next, the array with the adhering agarose blocks containing the biological material is placed in the vertically oriented, plastic chamber (2), which is then filled with an appropriate solution and equilibrated to the required temperature using the temperature stabilisation module (8, 9, 10). In order to increase the diffusion of components of the incubating solutions to and from the agarose, the array may be mechanically agitated in the incubating solution or the entire incubation chamber may be agitated (2) or the incubation solution may be mixed or pumped. The incubation time and solution temperature may be controlled manually, or with the aid of a programmable control instrument (11). Following the completion of a series of such incubations in appropriate buffers designed to purify DNA, the multiple array or selected arrays may be stored in an appropriate solution at a reduced temperature for many months. DNA contained in blocks adhering to selected arrays or multiple arrays are suitable for restriction endonuclease digestion and/or for use in electrophoretic separation of nucleic acid fragments contained in the agarose, either immediately following the isolation of DNA or after a period of storage. Preferentially, the electrophoresis is PFGE.

The restriction endonuclease digestion of the DNA consists of the incubation of the blocks containing the purified DNA, attached to the array, several arrays or to the multiple array in an appropriate solution containing a restriction endonuclease or a number of restriction endonucleases. The digestion may be performed in the same chamber in which the DNA isolation was performed, or in a different chamber. Preferentially, the chamber is adapted in size to the number of arrays undergoing digestion, since the amounts of solution can then be limited which means a saving in the costs of restriction endonucleases used.

### EXAMPLE 2

### Construction of the Array for the Simultaneous Incubation of a Series of Biological Samples Embedded in Agarose

FIG. 2 A represents a projection of the array designed to hold agarose blocks with embedded biological material during the isolation of genomic DNA and restriction endonuclease digestion. For the purposes of illustration, round apertures were drawn. These may be of an arbitrary shape, such as regular or irregular polyhedrons, ellipses or other figures. In horizontal cross-section, the shape of these edges may be arbitrary, though preferentially, these edges are differentiated such that the ensure stable contact in between the agarose and array throughout the whole procedure.

FIG. 2B represents a projection of a series of arrays from FIG. 2A joined into a multiple array, facilitating the isolation and digestion of DNA from a multiple number of biological materials or the purification and digestion of DNA from many replicants of one biological material.

### EXAMPLE 3

### The Genotyping of Strains of Haemophilus influenzae Using the Classic Method of Incubating Agarose Blocks in Test-Tubes, as well as According to Present Invention

Cells of Haemophilus influenzae strains isolated from clinical material underwent DNA purification, restriction endonuclease hydrolysis and separation in an agarose gel according to a previously described protocol (Tarasi, A., D'Ambrosio, F., Perrone, G., Pantosti, A. 1998 Susceptibility and genetic relatedness of invasive Haemophilus influenzae type b in Italy. Microb Drug Resist. 4(4):301-6). DNA fragments digested with the SmaI restriction endonuclease were separated in a 1% agarose gel (Pulsed Field Certified Agarose, BIORAD, USA) in a CHEF-DR II System or CHEF-DR III System (BIORAD, USA), stained in an aqueous solution of ethidium bromide and illuminated with UV light. The image of fluorescing fragments was digitally stored using a UVP Gel Documentation System (UVP, USA) and the Grab-IT 2.55 software package.

FIG. 3, panel A represents an image of a gel, in which lanes 1 to 12 contain PFGE-separated genomic DNA fragments from 12 different strains of Haemophilus influenzae obtained with the method according to the present invention.

FIG. 3, panel B represents an image of a gel, in which lanes 1 to 12 contain PFGE-separated genomic DNA fragments from 12 different strains of Haemophilus influenzae obtained with the classic method of rinsing and incubation of agarose blocks with biological samples in various solutions in test-tubes.

On the basis of a comparison between both panels, it may be stated that genomic DNA isolated from cells and digested using the classic method and the method according to present invention yield analogous end results.

### EXAMPLE 4

### The Genotyping of Neisseria meningitidis Strains Using the Classic Method of Incubating Agarose Blocks in Test-Tubes, as well as According to Present Invention

Cells of Neisseria meningitidis strains isolated from clinical material underwent DNA purification, restriction endonuclease hydrolysis and separation in an agarose gel according to a previously described protocol (Verdu, M. E., Coll, P., Fontanals, D., March, F., Pons, I., Sanfeliu, I., Prats, G. 1996. Endemic meningococcal disease in Cerdanyola, Spain, 1987-93: molecular epidemiology of the isolates of Neisseria meningitidis. Clin Microbiol Infect. 2(3), 168-178.). DNA fragments digested with the BglII restriction endonuclease were separated in a 1% agarose gel (Pulsed Field Certified Agarose, BIORAD, USA) in a CHEF-DR II System or CHEF-DR III System (BIORAD, USA), stained in an aqueous solution of ethidium bromide and illuminated with UV light. The image of fluorescing fragments was digitally stored using a UVP Gel Documentation System (UVP, USA) and the Grab-IT 2.55 software package.

FIG. 4, panel A represents an image of a gel, in which lanes 1 to 12 contain PFGE-separated genomic DNA fragments from 12 different strains of Neisseria meningitidis obtained with the method according to the present invention.

FIG. 4, panel B represents an image of a gel, in which lanes 1 to 12 contain PFGE-separated genomic DNA fragments from 12 different strains of Neisseria meningitidis obtained with the classic method of rinsing and incubation of agarose blocks with biological samples in various solutions in test-tubes.

On the basis of a comparison between both panels, it may be stated that genomic DNA isolated from cells and digested using the classic method and the method according to present invention yield analogous end results.

### EXAMPLE 5

### The Genotyping of Staphylococcus aureus Strains Using the Classic Method of Incubating Agarose Blocks in Test-Tubes, as well as According to Present Invention

Cells of Staphylococcus aureus bacterial strains isolated from clinical material underwent DNA purification, restriction endonuclease hydrolysis and separation in an agarose gel according to a previously described protocol (Chung, M., de Lencastre, H., Matthews, P., Tomasz, A., Adamsson, I., Aires de Sousa, M., Camou, T., Cocuzza, C., Corso, A., Couto, I., Dominguez, A., Gniadkowski, M., Goering, R., Gomes, A., Kikuchi, K., Marchese, A., Mato, R., Melter, O., Oliveira, D., Palacio, R., Sa-Leao, R., Santos Sanches, I., Song, J. H., Tassios, P. T., Villari, P.; Multilaboratory Project Collaborators. 2000. Molecular typing of methicillin-resistant Staphylococcus aureus by pulsed-field gel electrophoresis: comparison of results obtained in a multilaboratory effort using identical protocols and MRSA strains. Microb Drug Resist. 6(3), 189-98.). DNA fragments digested with the SmaI restriction endonuclease were separated in a 1% agarose gel (Pulsed Field Certified Agarose, BIORAD, USA) in a CHEF-DR II System or CHEF-DR III System (BIORAD, USA), stained in an aqueous solution of ethidium bromide and illuminated with UV light. The image of fluorescing fragments was digitally stored using a UVP Gel Documentation System (UVP, USA) and the Grab-IT 2.55 software package. FIG. 5, panel A represents an image of a gel, in which lanes 1 to 12 contain PFGE-separated genomic DNA fragments obtained with the method according to the present invention.

FIG. 5, panel B represents an image of a gel, in which lanes 1 to 12 contain PFGE-separated genomic DNA fragments obtained with the classic method of rinsing and incubation of agarose blocks with biological samples in various solutions in test-tubes. On the basis of a comparison between both panels, it may be stated that genomic DNA isolated from cells and digested using the classic method and the method according to present invention yield analogous end results.

### EXAMPLE 6

### The Genotyping of Streptococcus pneumoniae Strains Using the Classic Method of Incubating Agarose Blocks in Test-Tubes, as well as According to Present Invention

Cells of Streptococcus pneumoniae bacterial strains isolated from clinical material underwent DNA purification, restriction endonuclease hydrolysis and separation in an agarose gel according to a previously described protocol (Lefevre, J. C., Faucon, G., Sicard, A. M., Gasc, A. M. 1993 DNA fingerprinting of Streptococcus pneumoniae strains by pulsed-field gel electrophoresis. J Clin Microbiol. 31(10), 2724-8.). DNA fragments digested with the SmaI restriction endonuclease were separated in a 1% agarose gel (Pulsed Field Certified Agarose, BIORAD, USA) in a CHEF-DR II System or CHEF-DR III System (BIORAD, USA), stained in an aqueous solution of ethidium bromide and illuminated with UV light. The image of fluorescing fragments was digitally stored using a UVP Gel Documentation System (UVP, USA) and the Grab-IT 2.55 software package. FIG. 6, panel A represents an image of a gel, in which lanes 1 to 12 contain PFGE-separated genomic DNA fragments obtained with the method according to the present invention.

FIG. 6, panel B represents an image of a gel, in which lanes 1 to 12 contain PFGE-separated genomic DNA fragments obtained with the classic method of rinsing and incubation of agarose blocks with biological samples in various solutions in test-tubes. On the basis of a comparison between both panels, it may be stated that genomic DNA isolated from cells and digested using the classic method and the method according to present invention yield analogous end results.

1. A method of increasing the percentage composition of nucleic acids among the general content of substances of cellular origin placed in a medium facilitating the contact between the cells and their components with the components of a solution surrounding the medium, characterised in that this medium is affixed to a solid medium during the performance of this procedure.

2. A method of the enzymatic, chemical or physical alteration of a nucleic acids structure obtained using the method according to claim 1, characterised in that the medium in which the nucleic acid is embedded during the procedure is affixed to a solid medium.

3. A method according to claim 1 characterised in that samples of different cells embedded in separate portions of the medium are simultaneously placed in one portion of a solution.

4. A method according to claim 2 characterised in that nucleic acids from samples of different cells found in separate portions of the medium are simultaneously placed in one portion of a solution.

5. A method according to claim 1 characterised in that in the solid medium there are spaces which are filled with the medium containing the cells.

6. A method according to claim 1, characterised in that the nucleic acid is DNA.

7. (canceled)

8. A method according to claim 1, characterised in that the nucleic acid is RNA.

9. (canceled)

10. A method according to claim 1 characterised in that the cells are bacterial cells.

11. A method according to claim 1 characterised in that the cells are bacterial cells belonging to one of the following genera: Staphyloccous, Streptococcus, Enterococcus, Escherichia, Klebsiella, Enterobacter, Serratia, Citrobacter, Proteus, Providencia, Morganella, Moraxella, Salmonella, Shigella, Helicobacter, Vibrio, Bacillus, Campylobacter, Yersinia, Pseudomonas, Acinetobacter, Stenotrophomonas, Aeromonas, Burkholderia, Neisseria, Haemophilus, Listeria, Mycobacterium, Bordetella, Legionella, Pasteurella, Clostridium, Weisella, Pediococcus, Erwinia, Brachyspira, Lactobacillus, Lactococcus, Leuconostoc, Bifidobacterium, Arcobacter, Treponema, Photobacterium Erysipelothrix, Rhizobium, Taylorella, Porphyromonas, Arthrobacter, Microbacterium, Micrococcus, Anaplasma, Leptospira, Rhodococcus or their mixture of arbitrary composition.

12. A method according to claim 1 characterised in that the medium is a chemical compound or mixture thereof in arbitrary proportions, such that it mechanically restrains cells introduced into it and enables them to maintain contact with the components of the solution surrounding it.

13. A method according to claim 2 characterised in that the medium is a chemical compound or mixture thereof in arbitrary proportions, such that it mechanically restrains nucleic acids introduced into it and enables them to maintain contact with the components of the solution surrounding it.

14. A method according to claim 1 characterised in the medium is agarose.

15. (canceled)

16. A method according to claim 1 characterised in that the medium is acrylamide.

17. (canceled)

18. A method according to claim 1 characterised in that the medium is a mixture of acrylamide and agarose in arbitrary proportions.

19. A method according to claim 2 characterised in that the medium is a mixture of acrylamide and agarose in arbitrary proportions.

20. A method according to claim 7 characterised in that changes in DNA structure are the results of its hydrolysis as a result of the activity of one or many restriction endonucleases.

21. A method according to claim 1 characterised in that the medium remains affixed to a solid medium throughout the whole duration of increasing the percentage composition and alteration of the structure of the nucleic acid.

22. A device for increasing the nucleic acid percentage composition among the total composition of substances of cellular origin as well as altering nucleic acid structure according to the method described in claim 1, consisting of:
(1) a module for stabilising temperature, which is comprised of a thermal shield (1), a temperature measuring/regulating system (8), a heating/cooling element (9) and a temperature sensor (10)
(2) a module for the incubation of cells, their lysis products and purified DNA contained in medium affixed to a solid medium, which includes: (2) an incubation chamber, one or more, e.g. of varying sizes, the array (3) facilitating the simultaneous preparation of a series of samples, a hose system (6), valves (7), solution chambers (4) and a pump (5). This module can be equipped with a device which facilitates the mechanical mixing of solutions during incubation, as well as a system for the automatic transfer of the array with samples through a sequence of incubation chambers (2a, 2b itd.)
(3) a control module (11) facilitating the control of the sequence and duration of each stage of the preparation of macromolecule preparation as well as of the temperatures of the solutions used during the incubation.

23. A device according to claim 22, characterised in that tasks performed during its operation are regulated by a system which is programmable.

24. A device according to claim 22, characterised in that the solid medium is a plate with numerous apertures of arbitrary shape and cross-section, in which the medium containing cells is placed.
